(19) United States
(12) Patent Application Publication
(10) Pub. No.: US 2014/0260570 A1
(43) Pub. Date: Sep. 18, 2014

(54) CONCRETE STRUCTURE MOISTURE MEASUREMENT SYSTEM

(71) Applicant: Amphenol Corporation, (unknown)

(72) Inventor: Christopher James Elliott Ranwell, Andover, MA (US)

(73) Assignee: Amphenol Corporation

(21) Appl. No.: 13/834,453

(22) Filed: Mar. 15, 2013

(51) Int. Cl. G01N 33/38

(52) U.S. Cl. CPC G01N 33/383
USPC 73/73

(57) ABSTRACT

A sensor assembly is provided for measuring moisture content of concrete. The sensor assembly includes a sensor housing that is received within an opening in the concrete. The sensor housing includes an internal bore. The sensor housing is length adjustable so as to match a length of the opening. The sensor assembly further includes a moisture sensor received within the internal bore of the sensor housing. The sensor measure the moisture content of the concrete.

### BACKGROUND OF THE INVENTION

1. Field of the Invention

The present invention relates generally to a moisture sensor assembly and, in particular, to a height adjustable sensor assembly for measuring moisture content.

2. Discussion of the Prior Art

A moisture sensor is used to detect moisture content of a material, such as a structure or concrete slab. The moisture sensor is supported by a sensor housing, with the sensor housing being inserted into an opening in the concrete slab. The moisture sensor can then measure the moisture content within the concrete of the slab.

According to certain standards, moisture content should be measured at a specific depth of each concrete slab. In one example, according to the American Society for Testing and Materials (ASTM) standard F2170-11, moisture content is to be measured at a depth of 40% of the thickness of each respective concrete slab.

Due to thickness variations of the concrete in different slabs, this 40% depth may vary among the different slabs of concrete. For instance, in some examples, a thickness of the concrete slabs can vary between 10.1 centimeters (˜4 inches) and 15.3 centimeters (˜6 inches). Due to the varying depths required for moisture measurement in the concrete, varying sizes (e.g., lengths) of sensor housings are also needed for supporting the respective moisture sensors at the appropriate depth (e.g., 40% of depth). In the past, sensor housings had fixed lengths, such that a different sensor housing (of a different length) was required for each concrete thickness. Also in the past, a single fixed length sensor housing was used and the sensor housing was individually and manually cut down to a size for a specific concrete slab site. There is a need and benefit for a sensor housing to be length-adjustable so as to support the moisture sensor within concrete slabs of varying thicknesses.

### BRIEF DESCRIPTION OF THE INVENTION

The following presents a simplified summary of the invention in order to provide a basic understanding of some example aspects of the invention. This summary is not an extensive overview of the invention. Moreover, this summary is not intended to identify critical elements of the invention nor delineate the scope of the invention. The sole purpose of the summary is to present some concepts of the invention in simplified form as a prelude to the more detailed description that is presented later.

In accordance with one aspect, the present invention provides a sensor assembly for measuring moisture content of concrete. The sensor assembly inchldes a sensor housing received within an opening in the concrete. The sensor housing includes an internal bore. The sensor housing is length adjustable so as to match a length of the opening. A moisture sensor is received within the internal bore of the sensor housing. The sensor is configured to measure the moisture content of the concrete.

In accordance with another aspect, the present invention provides a sensor assembly for measuring moisture content of concrete. The sensor assembly includes a sensor housing received within an opening in the concrete. The sensor housing includes a first housing portion and a second housing portion. The sensor housing is length adjustable by moving the first housing portion and the second housing portion with respect to each other such that a length of the sensor housing matches a length of the opening. At least one of the first and second housing portions define an internal bore of the sensor housing. A moisture sensor is received within the internal bore of the sensor housing. The sensor measures the moisture content of the concrete.

In accordance with another aspect, the present invention provides a sensor assembly including a sensor housing configured to be received within an opening in concrete. The sensor housing includes a first housing portion and a second housing portion movably attached with respect to the first housing portion. The first housing portion and the second housing portion are length adjustable with respect to each other so as to match a length of the opening in the concrete.

### BRIEF DESCRIPTION OF THE DRAWINGS

The foregoing and other aspects of the present invention will become apparent to those skilled in the art to which the present invention relates upon reading the following description with reference to the accompanying drawings, in which:

FIG. 1 is a perspective view of an example sensor assembly, which is in a partially exploded state, in accordance with an aspect of the present invention;

FIG. 2 is a sectional view of the sensor assembly including an example sensor housing supporting a sensor therewithin;

FIG. 3 is a view of a drill for creating a drill hole opening to a desired depth in a slab of concrete sectional;

FIG. 4 is a sectional view similar to FIG. 3 after the drill has created the drill hole opening to the desired depth in the concrete;

FIG. 5 is a sectional view similar to FIG. 4 in which the sensor housing is being inserted into the drill hole;

FIG. 6 is a sectional view similar to FIG. 5 in which the sensor housing is fully inserted into the drill hole and is being rotated to adjust the length of the sensor housing to match a length of the drill hole;

FIG. 7 is a sectional view similar to FIG. 6 in which the sensor is being inserted into the sensor housing by means of a sensor positioning device; and

FIG. 8 is a sectional view similar to FIG. 7 in which the sensor assembly is fully assembled with the sensor being inserted into the sensor housing.

### DETAILED DESCRIPTION OF THE INVENTION

Example embodiments that incorporate one or more aspects of the present invention are described and illustrated in the drawings. These illustrated examples are not intended to be a limitation on the present invention. For example, one or more aspects of the present invention can be utilized in other embodiments and even other types of devices. Moreover, certain terminology is used herein for convenience only and is not to be taken as a limitation on the present invention. Still further, in the drawings, the same reference numerals are employed for designating the same elements.

FIG. 1 depicts an example embodiment of a sensor assembly 10, in an exploded condition, in accordance with one aspect of the invention. It is to be appreciated that FIG. 1 merely shows one example of the possible structure/configuration of the sensor assembly 10, and that that other examples are contemplated within the scope of the present invention. In general, the sensor assembly 10 is used for monitoring moisture content within an environment.

The sensor assembly 10 can be used in any number of environments. For instance, the sensor assembly 10 can be used in residential locations (e.g., houses, basements, attics, etc.), commercial locations, indoor/outdoor locations, or the like. In one possible example, the sensor assembly 10 is placed within an opening 12 in a slab of concrete 14. While the discussion presented for the sensor assembly 10 is in association with concrete 14, it should be appreciated that the sensor assembly 10 should not be construed to be limited based upon the concrete. Concrete should be broadly construed in that the sensor assembly 10 could be placed within an opening in any number of composite construction materials (e.g., aggregates, cements, etc.). Herein the slab or structure of concrete 14, regardless of its composite construction/materials is simply referred to as concrete.

The sensor assembly 10 includes a sensor housing 20 that is positionable within the opening 12 of the concrete 14. It is to be appreciated that the sensor housing 20 is shown in a partially exploded state detached from the concrete 14 in FIG. 1 for illustrative purposes and to more clearly depict portions of the sensor housing 20. In operation, however, the sensor housing 20 will be in a fully assembled state and positioned within the opening 12. The sensor housing 20 can be formed of any number of materials, including plastics (e.g., polypropylene, etc.), metals, or the like.

The sensor housing 20 includes a first housing portion 22. The first housing portion 22 is an elongated housing that extends between a first end 24 and an opposing second end 26. In this example, the first housing portion 22 is shown as being elongate and having a generally circular cross-sectional shape. In other examples, the first housing portion 22 can be longer or shorter in length and/or larger or smaller in cross-sectional size (e.g., diameter). Accordingly, the first housing portion 22 shown in FIG. 1 represents merely one of any number of possible structures/configurations for the first housing portion 22. The first housing portion 22 is positionable to extend at least partially into (i.e., be located at least partially within) the opening 12 in the concrete 14.

The first housing portion 22 includes a male threading 28. The male threading 28 extends helically/spirally around an outer surface of the first housing portion 22. The male threading 28 can extend at least partially along the length of the first housing portion 22. For instance, in the shown example, the male threading 28 extends from the first end 24 towards the second end 26 of the first housing portion 22. In other examples, the male threading 28 could extend a longer or shorter distance than as shown. It is to be appreciated that the first housing portion 22 is not limited to the male threading 28 of FIG. 1, and in other examples, could include any number of attachment structures, such as snap fit means, mechanical fasteners, or the like.

The first housing portion 22 can include a length indicia 30 positioned along the length of the first housing portion 22 from the first end 24 towards the second end 26. The length indicia 30 can be displayed in metric system units (e.g., millimeters, centimeters, etc.) or U.S. customary units (e.g., inches). The length indicia 30 will indicate, in one example, a total length of the sensor housing 20, which, as will be described below, can be varied based on a thickness of the concrete 14. In the shown example, the length indicia 30 includes length markings or other similar symbols indicating the length of the sensor housing 20. In another example, the length indicial 30 could indicate an approximate location/depth of a sensor positioned within the sensor housing 20.

Turning now to the second end 26, the first housing portion 22 further includes a retaining portion 34 disposed at the second end 26. The retaining portion 34 defines a larger cross-sectional size than a cross-sectional size of the first housing portion 22. Indeed, the retaining portion 34 can function as a shoulder, stop, or the like. In one example, the retaining portion 34 has a larger cross-sectional size than a cross-sectional size of the opening 12. As such, the retaining portion 34 will limit movement of the sensor assembly 10 with respect to the concrete 14 when the first housing portion 22 is positioned to extend at least partially into (i.e., be located at least partially within) the opening 12 in the concrete 14 and retaining portion 34 engages the concrete 14. In the shown example, the retaining portion 34 has a generally circular shape, though the retaining portion 34 is not so limited. In other examples, the retaining portion 34 could include an oval shape, quadrilateral shapes (e.g., square, rectangular, etc.), or nearly any type of shape/construction that is larger than the cross-sectional size of the opening 12.

The first housing portion 22 further includes an opening 36 disposed at the second end 26. The opening 36 is connected to an internal bore 38 that extends longitudinally through the first housing portion 22. In one example, the internal bore 38 extends completely through the first housing portion 22 from the first end 24 to the second end 26, such that the first housing portion 22 includes the opening 36 and an opposed opening (not shown) at the first end 24. In this example, the opening 36 and internal bore 38 are generally circularly shaped, though other shapes are envisioned. Further, the opening 36 and internal bore 38 include any variety of cross-sectional widths (e.g., diameter), such as by being larger or smaller than as shown.

The sensor housing 20 can further be provided with a cover 45 (illustrated in FIG. 1). The cover 45 can be attached to the opening 36. The cover 45 is selectively removable from the opening 36, such that the internal bore 38 can be accessed when the cover 45 is removed. In one example, the cover 45 limits debris (e.g., dust, dirt, moisture, etc.) from entering the sensor housing 20 when the cover 45 is attached to the opening 36.

The cover 45 can include a removal opening 46 allowing for the selective removal of the cover 45 from the opening 36 of the first housing portion 22. The removal opening 46 defines a hole, aperture, slot, or the like formed in the cover 45. In one example, the removal opening 46 is sized and shaped to receive a device that can pry the cover 45 loose from the first housing portion 22. This device can be, for example, a specialized tool, a screwdriver, a key, or the like. The removal opening 46 provides an ability to remove the cover 45 from the opening 36, but reduces the likelihood of the cover 45 being inadvertently removed from the opening 36.

It is to be appreciated that the cover 45, including the removal opening 46, includes only one of any number of means for covering the opening 36 in the sensor housing 20. Indeed, in other examples, the opening 36 could be permanently or temporarily be covered by an adhesive, glue, or other similar materials. Likewise, the sensor housing 20 is not limited to the specific size, shape, and structure of the cover 45, as other constructions of the cover 45 are envisioned.

The sensor housing 20 further includes a second housing portion 50. In FIG. 1, the second housing portion 50 is shown in a detached state from the first housing portion 22. In operation, however, the second housing portion 50 will receive the first housing portion 22 therewithin. Further, the second housing portion 50 is positionable to extend into (i.e., be located within) the opening 12 in the concrete 14.

The second housing portion 50 is an elongated housing that extends between a first end 52 and an opposing second end 54. In this example, the second housing portion 50 has a generally circular cross-sectional shape. In other examples, the second housing portion 50 can be longer or shorter in length and/or larger or smaller in cross-sectional size (e.g., diameter). Accordingly, the second housing portion 50 shown in FIG. 1 represents merely one of many possible structures/configurations for the second housing portion 50.

The second housing portion 50 includes one or more gripping portions 56. The gripping portions 56 extend circumferentially around an outer surface of the second housing portion 50. In this example, the one or more gripping portions 56 include three gripping portions, though any number of gripping portions are envisioned. For instance, two of the gripping portions are disposed towards the first end 52 while one of the gripping portions is disposed towards the second end 54 in FIG. 1. In other examples, however, the gripping portions 56 could be disposed at any number of locations along the second housing portion 50. The gripping portions 56 each define an enlarged cross-sectional size (e.g., diameter) than the remaining second housing portion 50. In operation, the gripping portions 56 can contact and/or engage walls of the opening 12 of the concrete 14. As such, the gripping portions 56 can limit inadvertent movement of the second housing portion 50 with respect to the concrete 14.

The second housing portion 50 further includes an internal bore 60. The internal bore 60 extends longitudinally through the second housing portion 50 between the first end 52 and the second end 54. While the internal bore 60 extends completely through the second housing portion 50 in this example, the internal bore 60 is not so limited. Rather, in other examples, the internal bore 60 may extend only partially along the length of the second housing portion 50, such as from the second end 54 towards the first end 52 but stopping short of the first end 52. In this example, the internal bore 60 has a generally circularly shaped cross-section to match the cross-sectional shape of the first housing portion 22. Likewise, the internal bore 60 is slightly larger in cross-sectional size than the first housing portion 22, so as to receive the first housing portion 22 therewithin. In other examples, however, the internal bore 60 could be larger or smaller in size than as shown, so as to accommodate for different sizes of the first housing portion 22.

The second housing portion 50 includes a female threading 62. The female threading 62 extends circumferentially around an inner surface that defines the internal bore 60. The female threading 62 extends at least partially along the length of the second housing portion 50 between the first end 52 and the second end 54. For instance, in the shown example, the female threading 62 extends from the first end 52 of the second housing portion 50 towards the second end 54. The female threading 62 can match a size/shape of the male threading 28 such that the female threading 62 and male threading 28 can engage and mate with each other.

When the first housing portion 22 is inserted into the second housing portion 50, the male threading 28 will threadingly engage the female threading 62. Rotation of one of the first housing portion 22 and second housing portion 50 with respect to the other will cause movement of the first housing portion 22 with respect to the second housing portion 50. For instance, if the first housing portion 22 is moved (e.g., rotated) with respect to the second housing portion 50, the first housing portion 22 and second housing portion 50 will linearly move with respect to each other. Likewise, if the second housing portion 50 is moved (e.g., rotated) with respect to the first housing portion 22, the first housing portion 22 and second housing portion 50 will linearly move with respect to each other. Rotation in one direction (e.g., clockwise or counter-clockwise) can cause linear extension of the first housing portion 22 with respect to the second housing portion 50. Rotation in an opposing direction (e.g., clockwise or counter-clockwise) can cause linear retraction of the first housing portion 22 with respect to the second housing portion 50.

Turning now to FIG. 2, a non-exploded, sectional view of the sensor assembly 10 along line 2-2 of FIG. 1 is shown. It is to be appreciated that in FIG. 2, the sensor assembly 10 is shown in a fully assembled view as opposed to the partially exploded view of FIG. 1. Indeed, in FIG. 2, the first housing portion 22 is mated/engaged with the second housing portion 50. This fully assembled can be inserted into the opening 12 of the concrete 14.

Referring still to the internal bore 60, the second housing portion 50 further includes a shoulder 64. The shoulder 64 defines an inward projection that extends towards a center of the second housing portion 50. The shoulder 64 therefore defines a reduced cross-sectional size of the internal bore 60. In one example, the shoulder 64 forms a generally circular cross-sectional shape, though other shapes (e.g., oval, quadrilateral, etc.) are envisioned. The shoulder 64 will extend longitudinally at least partially along a length of the second housing portion 50. In the shown example, the shoulder 64 is positioned in closer proximity to the first end 52 of the second housing portion 50. In other examples, however, the shoulder 64 is not so limited to this location, and, indeed, could be located closer to the first end 52 or second end 54 than as shown and/or extend a longer or shorter distance.

The sensor assembly 10 further includes a moisture sensor 70. The sensor 70 can be positioned within the sensor housing 20. It is to be appreciated that the sensor 70 shown in FIG. 2 includes only one of many possible sensors. Indeed, in other examples, the sensor 70 can be larger or smaller in size than as shown, and may include a variety of different constructions, structures, or the like. As such, the sensor 70 depicted in FIG. 2 is not intended to be limiting.

The sensor 70 can detect one or more conditions within the concrete 14. In one example, the sensor 70 can detect a moisture level/content of the concrete 14. As such, in one example, the sensor 70 includes a moisture content sensor. The sensor 70 is of course not limited to any specific type/mode of detecting moisture. Also in other examples, the sensor 70 could additionally detect temperature, humidity, or the like.

The example sensor 70 is an elongated, generally linearly extending structure extending between a first sensor end 72 and an opposing second sensor end 74. The sensor 70 has a cross-sectional size (e.g., diameter) that is sized so as to be received within the internal bore 60 of the second housing portion 50. In this example, the cross-sectional size of the sensor 70 is smaller than the cross-sectional size of the internal bore 60, such that the sensor 70 can be received therewithin. In other examples, the sensor 70 is not limited to this size, and could be longer/shorter in length, or the like. In one example, at least a portion of the sensor can be placed at 40% of the depth of the concrete 14. For instance, the second sensor end 74 can be positioned at the 40% depth, though in other examples, other portions of the sensor 70 could similarly be positioned at the 40% depth.

In one example, the sensor 70 can be placed within the sensor housing 20 at nearly any depth while accurately detecting moisture content of the concrete 14. In particular, since the sensor housing 20 includes the cover 45, the sensor housing 20 can be generally closed and sealed with the cover 45 once the sensor 70 is positioned within the sensor housing 20. The sensor 70 can then be left within the sensor housing 20 for a period of time, such as, in one example, an hour, or, in another example, 72 hours, or any other time limits. During this time, the conditions (e.g., moisture) will stabilize throughout the sensor housing 20, such that the sensor 70 can measure the stabilized moisture content of the surrounding concrete 14. Accordingly, the sensor 70 can accurately measure the moisture content of the concrete 14 whether the sensor 70 is located closer to the second end 26 or the first end 52.

The sensor 70 includes a sensor port 76 disposed at the first sensor end 72. It is to be appreciated that the sensor port 76 is not limited to this location, and, instead, could be positioned at any number of locations along the sensor 70. The sensor port 76 defines an output port for transmitting information related to the one or more conditions to a secondary/auxiliary device. In one example, the sensor port 76 can be attached (e.g., through wires, or the like) to a moisture meter (not shown). In such an example, information related to the moisture level/content of the concrete 14 will be detected by the sensor 70. This information can then be transmitted through the sensor port 76, through an attachment means (e.g., wires or the like), and to the moisture meter. The sensor port 76 allows for selective attachment/detachment of the attachment means. As such, when the sensor 70 is not transmitting information to the moisture meter, the attachment means can be detached from the sensor 70.

The sensor 70 further includes at least one channel 78. The channel 78 defines a groove, gap, opening, indentation, or the like extending circumferentially around an outer surface of the sensor 70. The channel 78 in this example is disposed towards the second sensor end 74 of the sensor 70, though other locations along the length of the sensor 70 are envisioned. Further, while the channel 78 is shown to include two channels in this example, in other examples, any number of channels (e.g., one or more) are envisioned.

The sensor assembly 10 further includes one or more engagement members 80 for engaging and contacting each of the sensor 70 and the second housing portion 50. The engagement members 80 extend circumferentially around the sensor 70. In one example, the engagement members 80 include an elastically deformable material, such as rubber or the like. In such an example, the engagement members 80 can include O-rings, or the like.

The engagement members 80 can be positioned within the channel 78 in contact/engagement with an outer surface of the sensor 70. In this example, the engagement members 80 have an inner diameter that is slightly larger than a diameter of the sensor 70, such that the engagement members 80 are received within the channel 78 of the sensor 70. The engagement members 80 will contact the shoulder 64 of the second housing portion 50 on an outer surface of the engagement members 80. Due to the engagement members 80 contacting the channel 78 on one side and the shoulder 64 on an opposing side, the engagement members 80 can be compressed between the sensor 70 and the second housing portion 50. This compression will tend to hold the sensor 70 in place with respect to the second housing portion 50 and limit movement of the sensor 70. It is to be appreciated that the engagement members 80 also allow for selective removal of the sensor 70 from the second housing portion 50.

Turning now to FIG. 3 to 8, an example operation of measuring moisture content of the concrete 14 with the sensor assembly 10 will now be described. It is to be appreciated that FIGS. 3 to 8 depict only a portion of the concrete 14. In other examples, the concrete 14 can be larger or smaller than as shown (e.g., fully formed concrete slabs) and/or thicker or thinner. Accordingly, the dimensions of the concrete 14 shown in FIGS. 3 to 8 are not to scale and are not intended to depict the exact length/thickness of the concrete 14.

Referring to FIG. 3, the concrete 14 in this example has a thickness 84. The thickness 84 of the concrete 14 includes any number of dimensions. For instance, in one example, the concrete 14 can have a thickness 84 of between approximately 10.1 centimeters (˜4 inches) and 15.3 centimeters (˜6 inches). Of course, other thickness dimensions are envisioned. In accordance with standard ASTM F2170-11, moisture of the concrete 14 is to be measured by drilling a hole to a depth of 40% of the thickness of the concrete 14. Accordingly, for a slab of concrete 14 having a thickness 84 of 10.1 centimeters (˜4 inches), the depth of the opening 12 drilled into the concrete 14 should be approximately 4.04 cm (˜1.6 inches). Similarly, for a slab of concrete 14 having a thickness 84 of 15.3 centimeters (˜6 inches), the depth of the opening 12 drilled into the concrete 14 should be approximately 6.12 cm (˜2.4 inches). As such, in one example, a portion of the sensor 70, positioned within the sensor housing 20, can be located at the 40% depth of the concrete 14. As will be described below, the second sensor end 74 of the sensor 70 can be positioned at this 40% depth. The opening 12, in one example, is approximately 19 millimeters (0.75 inches) diameter, though other diameters are also envisioned.

It is to be appreciated that the dimensions of FIGS. 3 and 4 are not drawn to scale. For instance, referring briefly to FIG. 4, the thickness 84 of the concrete 14 and the 40% depth (illustrated as a depth 104) are likewise not drawn to scale. In operation, however, the depth 104 will comprise approximately 40% of the length of the thickness 84.

To create the opening 12 of a desired depth, a drill 90 can be used. The drill 90 in FIG. 3 is somewhat generically/schematically depicted, as it is to be appreciated that the drill 90 can include any number of constructions. In this example, the drill 90 includes a cutting tool 92. The cutting tool 92 includes any number of structures that create an opening/hole in the concrete 14. For instance, the cutting tool 92 can include drill bits, driver bits, or the like. In this example, the cutting tool 92 includes a twist drill bit with one or more helical flutes extending along a shaft of the cutting tool 92. Of course, the cutting tool 92 is not limited to this construction, and includes any number of sizes, shapes, and constructions that will create the opening/hole in the concrete 14.

The drill 90 further includes a stop 94 that allows for selective adjustment of a length of the cutting tool 92. The stop 94 can include, for example, a chuck, or other similar device for supporting the cutting tool 92. The cutting tool 92 can be moved with respect to the stop 94 so as to adjust the total length of the cutting tool 92. In operation, the depth of the opening 12 formed in the concrete 14 depends on the thickness 84 of the concrete 14. For instance, as set forth above, the depth of the opening 12 in one example is approximately 4.04 cm (˜1.6 inches) while in another example is approximately 6.12 cm (˜2.4 inches). As such, the stop 94 will allow for a desired depth of the opening 12 in the concrete 14 to be achieved.

To create the opening 12, the drill 90 is moved in a first direction 100. As shown, the drill 90 moves in the first direction 100 towards the concrete 14. The cutting tool 92 will contact and engage the concrete 14, thus creating the opening 12. The drill 90 can continue to move in the first direction 100 until the stop 94 contacts the concrete 14, thus indicating that the opening 12 having a desired depth in the concrete 14 has been achieved. In another example, the drill 90 will move in the first direction 100 until the desired depth in the concrete 14 is reached. At that point, the drill 90 can be removed from the concrete 14.

Turning now to FIG. 4, the concrete 14 is shown after the drill 90 has been removed. In this example, a drill hole 102 including the opening 12 has been formed. The drill hole 102 includes the depth 104. It is to be appreciated that the drill hole 102 and the depth 104 are not shown to scale in FIG. 4. Rather, the drill hole 102 and depth 104 are shown to be slightly larger in size than in operation for ease of illustration. In other examples, however, the drill hole 102 may have the depth 104 that is smaller than as shown. Indeed, in one example, according to ASTM F2170-11, the depth 104 of the drill hole 102 will be approximately 40% of the thickness 84 of the concrete 14.

Turning now to FIG. 5, the sensor housing 20 can be inserted into the drill hole 102. In particular, the sensor housing 20 is inserted through the opening 12 and into the drill hole 102 by moving in the first direction 100. In the shown example, the length of the sensor housing 20 is initially adjusted prior to insertion into the drill hole 102. As such, the first housing portion 22 and second housing portion 50 are rotated with respect to each other such that the length of the sensor housing 20 generally matches the depth 104 of the drill hole 102. In other examples, however, the sensor housing 20 can be inserted into the drill hole 102 prior to a length adjustment of the sensor housing 20. In such an example, the first housing portion 22 and second housing portion 50 will be rotated with respect to each other to adjust the length after the sensor housing 20 is inserted into the drill hole 102.

Turning now to FIG. 6, the sensor housing 20 can further be length adjusted after being received within the drill hole 102. In particular, the first housing portion 22 is rotated in a second direction 110 with respect to the second housing portion 50. Due to the gripping portions 56 contacting and engaging walls of the drill hole 102, the second housing portion 50 is generally limited from moving, rotating, etc. In one example, rotation in the second direction 110 causes the sensor housing 20 to shorten in length, such that the first housing portion 22 will be further received within the second housing portion 50. Of course, in other examples, rotation in the second direction 110 can instead cause the sensor housing 20 to enlarge in length. In either example, the first housing portion 22 will continue to be rotated in the second direction 110 until the retaining portion 34 contacts the concrete 14. Due to the retaining portion 34 contacting the concrete 14, unintended longitudinal movement of the sensor housing 20 with respect to the drill hole 102 is limited/reduced.

Turning now to FIG. 7, the sensor 70 can be positioned within the sensor housing 20. In particular, a sensor positioning device 120 is used to place (e.g., install) the sensor 70 within the sensor housing 20. The sensor positioning device 120 includes any number of different sizes, shapes, and constructions. In this example, the sensor positioning device 120 includes an elongated, linearly extending structure. The sensor positioning device 120 has a cross-sectional size that is small enough to fit within the sensor housing 20, in particular, the internal bore 38 of the first housing portion 22 and the internal bore 60 of the second housing portion 50. The sensor positioning device 120 can therefore be selectively inserted and removed from the sensor housing 20.

The sensor positioning device 120 includes a holding portion 122 disposed at an end of the sensor positioning device 120. The holding portion 122 is designed to selectively grip the sensor 70. The holding portion 122 includes, for example, a hook, catch, or other similar gripping structure for gripping the sensor 70. In one example, the sensor positioning device 120 is rotatable, such that the holding portion 122 can selectively grip or release the sensor 70.

The holding portion 122 can selectively grip a portion of the sensor 70. In one example, the sensor 70 includes an engagement portion 124 for engaging the holding portion 122. In the shown example, the engagement portion 124 includes a hook that is sized and shaped to mate with the holding portion 122. In other examples, however, the engagement portion 124 is not limited to the shown structure, and can include any number of constructions. The engagement portion 124 allows for selective engagement/gripping by the holding portion 122. As such, the sensor 70 can be selectively held/gripped or released by the sensor positioning device 120.

It is to be appreciated that the sensor 70 in FIG. 7 includes an engagement member 180 that is different in construction than the engagement member 80 of FIG. 2. In particular, the engagement member 180 in this example includes a single engagement member that extends circumferentially around the sensor 70. To accommodate for the engagement member 180, the second housing portion 50 includes a shoulder 164 that is different in construction than the shoulder 64 of FIG. 2. In particular, the shoulder 164 in this example is generally solid and not hollow. Of course, it is understood, that the sensor 70 and second housing portion 50 can include either of the engagement members 80, 180 or shoulders 64, 164, respectively, that are shown and described herein while functioning in a similar/identical manner.

In operation, the sensor positioning device 120 will initially grip/hold the sensor 70. In particular, the holding portion 122 of the sensor positioning device 120 grips the engagement portion 124 of the sensor 70. Next, the sensor positioning device 120 and sensor 70 are inserted into the sensor housing 20 (e.g., internal bores 38, 60) in the first direction 100. The sensor 70 is moved further into the sensor housing 20 until the engagement member 180 contacts the shoulder 164. With the engagement member 180 contacting the shoulder 164, the sensor 70 is generally limited from moving with respect to the sensor housing 20.

Next, the sensor positioning device 120 can be removed from the sensor housing 20. In particular, the sensor positioning device 120 can first be rotated in a third direction 130. Rotation in the third direction 130 will cause the holding portion 122 to become disengaged (and released) from the engagement portion 124. The sensor positioning device 120 can then be removed from the sensor 70 by moving the sensor positioning device 120 in a fourth direction 132 that is opposite from the first direction 100. With the sensor positioning device 120 having been removed, the sensor 70 will remain engaged within the sensor housing 20.

Turning now to FIG. 8, the sensor assembly 10 is shown with the sensor 70 placed/positioned within the sensor housing 20. As can be seen, the sensor 70 is relatively limited from being inadvertently moved or dislodged due to being housed within the sensor housing 20. Further, the cover 45 can be placed atop the sensor housing 20 to limit debris from entering the sensor housing 20. In operation, a user can leave the sensor 70 within the sensor housing 20 for a period of time, such as, in one example, an hour, or, in another example, 72 hours, or any other time limits. During this time, the conditions (e.g., moisture) can stabilize and the sensor 70 can measure the stabilized moisture content of the surrounding concrete 14. After the period of time has passed, the cover 45 can be removed, and the sensor 70 can be attached to an auxiliary device, such as a moisture meter. Accordingly, information related to the moisture content of the surrounding concrete 14 can then be transmitted to the moisture meter.

In the illustrated example, a portion of the sensor 70 is shown to be positioned at approximately 40% of the depth of the concrete 14. In particular, in this example, the second sensor end 74 is positioned at approximately 40% of the depth of the concrete 14. Accordingly, in the illustrated example, the second sensor end 74 can approximately match the location of the end of the second housing portion 50 at 40% of the depth of the concrete 14.

The sensor assembly 10, in particular the sensor housing 20, is length adjustable so as to match the length of the drill hole. Due to standards requiring moisture content to be measured at 40% of the depth of a concrete slab, a drill hole length will vary based on concrete slabs having different thicknesses. Accordingly, since the sensor housing 20 is length adjustable (e.g., between about 10 centimeters and 15.5 centimeters), the sensor housing 20 can accommodate for various thicknesses of concrete slabs. In particular, it is no longer necessary to provide sensor housings having fixed lengths. Rather, the sensor housing 20 can be adjusted in length to match the drill hole length.

The sensor assembly 10 may conveniently be packaged in the form of a kit that includes a number of components providing increased convenience, flexibility, and adaptability to operators in the field. In one example, the kit may include the sensor housing 20 that is received within the opening 12 in the concrete 14. The sensor housing 20 is length adjustable to as to match the length of the opening 12. The kit may further include the sensor 70 that can be received within the internal bore 38, 60 that extends within the sensor housing 20. The sensor 70 can measure the moisture content of the concrete 14.

The invention has been described with reference to the example embodiments described above. Modifications and alterations will occur to others upon a reading and understanding of this specification. Example embodiments incorporating one or more aspects of the invention are intended to include all such modifications and alterations insofar as they come within the scope of the appended claims.

What is claimed is:

1. A sensor assembly for measuring moisture content of concrete, the sensor assembly including:
a sensor housing configured to be received within an opening in concrete, the sensor housing including an internal bore, the sensor housing being length adjustable so as to match a length of the opening; and
a moisture sensor received within the internal bore of the sensor housing, the sensor being configured to measure a moisture content of the concrete.

2. The sensor assembly of claim 1, wherein the sensor housing includes a first housing portion and a second housing portion.

3. The sensor assembly of claim 2, wherein the first housing portion includes a male threading extending at least partially along an outer surface of the first housing portion.

4. The sensor assembly of claim 3, wherein the second housing portion includes a female threading extending at least partially along an inner surface of the second housing portion.

5. The sensor assembly of claim 4, wherein the male threading of the first housing portion engages the female threading of the second housing portion such that rotation of the first housing portion and the second housing portion with respect to each other adjusts a length of the sensor housing.

6. The sensor assembly of claim 1, wherein the sensor housing includes an opening disposed at an end of the sensor housing, the opening being in communication with the internal bore so as to receive the sensor therethrough.

7. The sensor assembly of claim 1, further including an engagement member positioned within the internal bore between the sensor and an inner surface of the sensor housing, the engagement member being configured to hold the sensor with respect to the sensor housing.

8. The sensor assembly of claim 7, wherein the engagement member extends circumferentially around the sensor in contact with each of the sensor and the inner surface of the sensor housing.

9. The sensor assembly of claim 1, wherein the sensor housing is length adjustable between 10 centimeters and 15.5 centimeters.

10. A sensor assembly for measuring moisture content of concrete, the sensor assembly including:
a sensor housing configured to be received within an opening in concrete, the sensor housing including a first housing portion and a second housing portion, the sensor housing being length adjustable by moving the first housing portion and the second housing portion with respect to each other such that a length of the sensor housing matches a length of the opening, at least one of the first and second housing portions defining an internal bore of the sensor housing; and
a moisture sensor received within the internal bore of the sensor housing, the sensor being configured to measure the moisture content of the concrete.

11. The sensor assembly of claim 10, wherein the first housing portion includes a male threading extending along an outer surface of the first housing portion, the male threading extending from a first end of the first housing portion towards an opposing second end.

12. The sensor assembly of claim 11, wherein the second housing portion includes a female threading extending at least partially along an inner surface of the second housing portion.

13. The sensor assembly of claim 12, wherein the male threading of the first housing portion engages the female threading of the second housing portion such that rotation of the first housing portion and the second housing portion with respect to each other adjusts a length of the sensor housing.

14. The sensor assembly of claim 13, wherein the sensor housing includes an opening disposed at the second end of the first housing portion, the opening being in communication with the internal bore so as to receive the sensor therethrough.

15. The sensor assembly of claim 10, further including an engagement member positioned within the internal bore between the sensor and an inner surface of the sensor housing, the engagement member being configured to hold the sensor with respect to the sensor housing.

16. The sensor assembly of claim 15, wherein the engagement member extends circumferentially around the sensor in contact with each of the sensor and the inner surface of the sensor housing.

17. The sensor assembly of claim 10, wherein the sensor housing is length adjustable prior to being inserted into the opening in the concrete.

18. The sensor assembly of claim 10, wherein the sensor housing is length adjustable after being inserted into the opening in the concrete.

19. A sensor assembly including:
a sensor housing configured to be received within an opening in concrete, the sensor housing including:
a first housing portion; and
a second housing portion movably attached with respect to the first housing portion, the first housing portion and the second housing portion being length adjustable with respect to each other so as to match a length of the opening in the concrete.

20. The sensor assembly of claim 19, wherein the first housing portion and the second housing portion define an internal bore, the internal bore being configured to receive a moisture sensor that is configured to measure a moisture content of the concrete.
